(19) United States
(12) Patent Application Publication
(10) Pub. No.: US 2023/0293063 A1
(43) Pub. Date: Sep. 21, 2023

(54) A PROPOFOL SENSOR

(71) Applicant: SOMNUS SCIENTIFIC LTD, Bath (GB)

(72) Inventors: David FERRIER, Bristol (GB); Ibidapo WILLIAMS, Bristol (GB); Janice KIELY, Bristol (GB); Richard LUXTON, Bristol (GB); Mark O’CONNELL, Bath (GB)

(21) Appl. No.: 17/927,209

(22) PCT Filed: May 17, 2021
(86) PCT No.: PCT/EP2021/063035

§ 371 Date: Nov. 22, 2022

(30) Foreign Application Priority Data
May 29, 2020 (GB) 2008081.8
Aug. 12, 2020 (GB) 2012582.9

(51) Int. Cl. A61B 5/145; C12Q 1/00; A61B 5/1477; A61B 5/1486; A61B 5/00; G01N 33/49

(52) U.S. Cl. CPC A61B 5/14546; A61B 5/1477; A61B 5/1486; A61B 5/4821; A61B 5/14528; A61B 5/14542; C12Q 1/005; C12Y 114/14001; G01N 33/4925; A61B 2505/05; A61B 2562/04

(57) ABSTRACT

An enzymatic electrochemical sensor for the detection of blood propofol is provided.

The present invention relates generally to methods, systems and apparatus for detecting and quantifying the intravenous anaesthetic propofol in solution.

Propofol (2,6-diisopropylphenol) is an intravenous drug used for the induction and maintenance of anaesthesia.

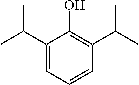

It has favourable characteristics, including rapid induction and a short half-life; consequently it has been the most commonly used intravenous anaesthetic for the last thirty years.

The most common practice in general anaesthesia is to use an intravenous anaesthetic, such as propofol, for the induction phase and volatile anaesthetics for the maintenance phase. However, it is possible to use propofol for both the induction and maintenance phases, in a process known as total intravenous anaesthesia (TIVA). There is a growing body of evidence of the advantages of TIVA over conventional volatile-based anaesthesia, including: reduced short-term side-effects, reduced cognitive effects, the potential for improved long-term survival for cancer patients, and a significantly reduced environmental impact.

Despite these numerous advantages, traditional volatile-based anaesthesia still accounts for the vast majority of administered general anaesthetics. The principle obstacle to a greater exploitation of TIVA is the lack of suitable methods for the continuous, real-time monitoring of blood propofol concentration in patients undergoing anaesthesia.

Established techniques for detection and quantification of propofol include high performance liquid chromatography (HPLC), which is used in conjunction with a variety of measurement techniques, with the most common being fluorimetric detection. Whilst it may be a ubiquitous technique, HPLC is not well suited to point-of-care applications due to its reliance on bulky and expensive equipment. Furthermore, HPLC offers only discrete, rather than continuous measurement. It also requires complex and time-consuming sample pre-treatment methods.

Mass spectrometry is another common technique for the detection and quantification of propofol in biological samples, in conjunction with either gas chromatography or liquid chromatography. As for HPLC, when analysing propofol in whole blood, serum or plasma, the propofol is extracted from the sample either by solvent or solid phase extraction. As for HPLC, the principle disadvantages of mass spectrometry techniques are the requirement for expensive and bulky equipment and the lack of capacity for continuous monitoring. A particular drawback is the requirement for lengthy analysis and sample preparation processes.

Research has been conducted into monitoring propofol in exhaled breath. However, as the relationship between blood propofol concentration and exhaled breath concentration is not fully understood, it is unclear whether this approach will be applicable to patient monitoring.

There are reports relating to the detection of propofol in urine. However, due to the significant time-lag between administering a drug and it or its metabolites presenting in the urine, this approach will not be applicable to real-time propofol monitoring during general anaesthesia. Whole blood, serum or plasma represent the most practical biological fluids for this application.

The present invention seeks to provide improvements in or relating to the detection and/or quantification of propofol.

Aspects and embodiments of the present invention relate to propofol monitoring.

Some aspects and embodiments provide, relate to or include discrete measurement of propofol, for example using a blood-gas analyser.

Some aspects and embodiments provide, relate to or include direct measurement of propofol, for example direct electrochemical measurement.

Some aspects and embodiments provide, relate or include real-time propofol monitoring

Some aspects and embodiments provide, relate to or include point-of-care propofol monitoring.

An aspect of the present invention provides a real-time, point-of-care, blood propofol concentration measurement sensor and/or measurement system.

Some aspects and embodiments relate to point-of-care monitoring of the blood concentration of propofol for general anaesthesia patients. This can include the use of a blood-gas analyser.

Some aspects and embodiments provide, relate to or include a solid-phase detection technique.

Some aspects and embodiments relate to a solution-phase propofol detection technique and its application to real-time propofol monitoring.

The aim of delivering point-of-care and/or real-time monitoring of blood propofol concentration during general anaesthesia places a number of requirements on any potential propofol sensing technique. For instance, any method must be capable of returning results within a sufficiently narrow window of time to provide information that is of practical use to anaesthetists or other healthcare professionals. This is a major reason why approaches such as HPLC and mass spectrometry, which have a time-to-results of the order of several tens of minutes at best, are of limited utility for this application. Any methods that require non-trivial sample pre-treatment will likely not be suitable for this same reason and, as such, sensors capable of functioning at physiological conditions are more suitable than those that are not (for instance optical techniques based on the Gibbs reaction, which requires alkaline conditions).

To be of use for patient monitoring in a surgical context, any sensor system needs to be capable of producing stable results over the duration of a surgical procedure, potentially eight hours or longer. Furthermore, it has been shown that propofol will slowly redistribute between the plasma and blood cell membranes over time, meaning that the time between the collection and measurement of a sample will need to be tightly controlled. As such, any technique for propofol monitoring (including real-time monitoring) must be suitable for automation, with minimal sample processing.

It is possible to detect propofol electrochemically, via its oxidation. Electrochemical approaches are attractive due to their potential for high sensitivity and ease of automation, but possess significant challenges in terms of specificity. Furthermore the oxidation reaction produces radicals which can undergo further reactions to produce polymeric molecules at the electrode surface, in a process referred to as electropolymerisation. These polymers are insoluble and non-conductive, and therefore lead to significant electrode fouling (also referred to as electrode passivation). Therefore, the direct electrochemical detection of propofol is currently not practical for real-world applications where any propofol sensor would be required to produce a stable current over periods of up to several hours.

It is likely during general anaesthesia that propofol will be co-administered with other drugs, and as such it is necessary that any propofol sensor possess a sufficient degree of specificity. Specificity is a particular challenge for electrochemical approaches as the potential window in which propofol is electrochemically oxidised corresponds to the electroactive window for many potential interfering compounds.

The sensor may be enzymatic.

The present invention also provides an enzyme-based electrochemical sensor for the detection of propofol.

An enzyme-based propofol sensor may be provided that avoids issues of electrode fouling, for example by converting the propofol into a quinone/quinol redox pair which can be detected via simple electrochemistry.

The sensor may comprise a cell, for example an electrode cell.

Some embodiments, for example, may include enzyme directly immobilised onto electrodes.

Some embodiments, for example, comprise a working electrode, a counter electrode and a reference electrode.

The electrode may, for example, comprise a two-electrode cell (with a working electrode and combined reference/counter electrode) or a three-electrode cell (with working, counter, and reference electrodes).

A cell may, for example, have two or three types of electrodes e.g. either working, combined reference and counter, reference or counter electrode. There can be multiples of each type within a cell.

Some embodiments may comprise multiple working electrodes within a single cell.

Some embodiments may comprise multiple cells on a sensor device.

Some embodiments may comprise two, three or more electrode cells.

A sensor may comprise a plurality of electrode cells. The cells may be “wired” or otherwise connected together, or they may be independent.

The counter electrode/s may be made of materials including but not limited to: carbon, gold, platinum, silver or silver/silver chloride.

The reference electrode/s may be made of materials including but not limited to silver and silver/silver chloride.

The working electrode/s may be made from materials including but not limited to carbon, gold, platinum, silver, copper, aluminium or indium tin oxide. The working electrode/s could either be used untreated or functionalised with nanomaterials in a manner such as described below, for example.

The working electrode/s could, for example, either be macro-scale (> 100 µm), micro-scale (1-100 µm) or nano-scale (< 1 µm) and could comprise of a single electrode or an array of multiple electrodes, either with all members of the array sharing the same counter electrode or with each member of the array having an associated, individual counter electrode.

The electrode/s could be fabricated using any appropriate technique such as screen printing or microfabrication techniques, including but not limited to photolithography, etching techniques or chemical vapour deposition.

The electrode/s may be planar or take the form of a nano-strip electrode, utilising passivation layers that can be fabricated from materials including but not limited to: silicon dioxide, silicon nitride or parylene.

The electrodes may comprise porous fritted material such as carbonaceous or something similar. These may function as coulometric electrochemical cells.

Some aspects and embodiments use ‘sensor redundancy’ to provide more reliable sensors. The signal from independent electrodes may be sampled and compared. Outliers are discarded to ensure failure in one electrode does not unduly influence the result.

Regarding electrode functionalisation, a film could, for example, be deposited directly onto the working electrode surface. Alternatively, prior to deposition, the surface could be functionalised with nanomaterials to improve the sensor performance.

A working electrode could be functionalised with a single layer of nanoparticles, with examples including but not limited to: iron oxide nanoparticles, gold nanoparticles, silver nanoparticles, platinum nanoparticles, copper nanoparticles, zinc oxide nanoparticles, nickel oxide nanoparticles, copper oxide nanoparticles, carbon nanoparticles, copper nanowires, carbon nanotubes or graphene nanosheets.

The working electrode could be functionalised with two or more layers of different nanoparticles, with examples including but not limited to: iron oxide nanoparticles, gold nanoparticles, silver nanoparticles, platinum nanoparticles, copper nanoparticles, zinc oxide nanoparticles, nickel oxide nanoparticles, copper oxide nanoparticles, carbon nanoparticles, copper nanowires, carbon nanotubes or graphene nanosheets.

The working electrode could be functionalised with single layers of nanomaterial composites with examples including but not limited to: carbon nanotubes functionalised with metal nanoparticles (with potential metals including but not limited to gold, silver, platinum or alloys thereof) or any combination of two or more of the following: carbon nanotubes, graphene nanosheets, gold nanoparticles, silver nanoparticles, platinum nanoparticles, copper nanoparticles, zinc oxide nanoparticles, nickel oxide nanoparticles, copper oxide nanoparticles or copper nanowires.

The working electrode could be functionalised with vertically aligned carbon nanotubes by plasma-enhanced chemical vapour deposition. These nanotubes can then be encapsulated in an insulating material such as epoxy or silicon dioxide to produce a nanoelectrode array.

The sensor may be based on a one or more members of the cytochrome P450 group of enzymes.

Cytochromes P450 are a group of heme-thiolate monooxygenases. In liver microsomes, this enzyme is involved in an NADPH-dependent electron transport pathway.

In one embodiment an electrochemical propofol sensor based upon the enzyme cytochrome P450 2B6 is provided. Cytochrome P450 2B6 belongs to a set of hepatic drug-metabolizing cytochromes.

The sensor may be based on an electrode, such as a screen-printed electrode.

In one embodiment deactivated yeast cells expressing the enzyme cytochrome P450 2B6 are immobilised, alongside gold nanoparticles, within a chitosan film upon the surface of a screen-printed electrode. In the presence of the cofactor NADP⁺ the enzyme converts propofol to a quinone/quinol redox pair that can be detected and quantified using simple electrochemistry. This approach avoids the issue of electrode fouling that commonly renders electrochemical propofol sensors impractical.

Currently there are known to be 18 super families of CYPP450 in humans, 43 subfamilies containing 57 genes and 59 pseudogenes. Primarily, these enzymes are expressed in the liver that are responsible for xenobiotic metabolism. Majorly, aromatic hydroxylation of propofol is mediated by CYP2C9 and CYP2B6, additional isoforms such as CYP2A6, CYP2C8, CYP2C18, CYP2C19 and CYPIA2 have been suggested. 90% of propofol is metabolized in the liver (Smits, Annaert and Allegaert, 2017).

CYP2B6 and CYP2C9 polymorphisms have shown significant metabolism on pro-drugs and individual variables. CYP2B6*6 allele and CYP2C9*2 allele with UGTIA9 and UGTIA6.

One or more enzyme isoforms (e.g. CYP2C9 and CYP2B6) may be use either independently or together, i.e. as may occur within the human liver. A combination of such enzyme elements may, for example, form a type of sandwich methodology.

In some embodiments the sensor brings together electrochemical sensing, CYP P450 and nanomaterials.

A lack of specificity of CYP enzymes could potentially cause problems. However, 2B6 is one of the less promiscuous enzymes. Furthermore, in some embodiments the action of 2B6 is not measured directly; therefore it is not enough that a potential interfering compound be a substrate of the enzyme - it must be converted into an electrochemically active molecule(s). Moreover, any potential interferant must be electrochemically active at or near the potential being measured to cause any problems (a case in point is ibuprofen, which contains a benzene ring but is not a substrate of 2B6 and is electrochemically active, but at a much higher potential and therefore causes no problems). Finally, any potential interferant must be present in quantities high enough to actually cause issues. Morphine, for example, is actually electrochemically active at the potential measured at in some embodiments, but its relative concentration to propofol means that it cannot be detected it, so it is not a problem.

Some aspects and embodiments are based on a principle that they do not use direct electron transfer between the electrode and the CYP enzyme. In contrast, by using an indirect method (via NADPH) and electrochemically detecting the reaction products the present invention can avoid the potential problems caused by the non-specific nature of CYP enzymes.

Some embodiments may include sensors modifiers. Variables such as, for example, pH and temperature may be used as sensor modifiers. It is reported that propofol undergoes hydroxyl substituent (—OH) on the benzene ring, which then becomes dissociated when the pH is acidic (<6.5) and positively charged in the solution.

Some embodiments use recombinant human CYP, P450 expressed within deactivated, permeabilised yeast cells. For example, CypExpress™ which is a product comprised of a specific, unmodified recombinant human CYP, P450 oxidoreductase cofactors, and antioxidants encapsulated in a semipermeable shell.

Some embodiments use recombinant human enzyme. Other embodiments use synthetic alternatives.

Some embodiments use: yeast cells and/or mammalian cells and/or bacterial cells and/or synthetic cells.

Enzymes including Cytochrome P450 2B6 (CYP2B6), CYP2C9, CYP2C19 or CYP2E1 can be expressed within deactivated, permeabilised yeast cells.

CYP enzyme activity requires a co-enzyme, such as cytochrome P450 reductase, and a cofactor such as NADH or NADPH, to be present to aid with electron transfer. This has been an impediment to the development of CYP enzyme biosensors until the development of “mediatorless” or direct biosensors which deliver electrons directly to the active site of the enzyme, containing iron-protoporphyrin IX (haem). Direct immobilisation of enzyme to an electrode surface has been shown to allow a direct measurement of CYP activity. A range of electrode surface materials have been reported as substrates for CYP biosensors, including gold, graphite and indium tin oxide (Schneider, 2013). Recently the application of nanostructures, such as gold nano-spheres, carbon nanotubes and graphene have been reported to enhance the sensitivity of enzyme biosensors through increased charge transfer (Preethichandra, 2019). None of the previous work examined the application of metal oxide nanoparticles to develop direct biosensors, nor had the use of CYP 2B6 been assessed with this method.

There have been no reports of the use of CYP2B6 or any other CYP enzyme for detection of propofol.

Some embodiments are based on immobilised CYP2B6 enzyme at an electrode surface to provide a sensitive, real-time measurement of propofol.

Some embodiments use cells expressing CYP2B6 enzyme.

A sensor could, for example, be prepared using yeast cells expressing a single CYP enzyme, a mixture of multiple different types of yeast cell — each expressing a different CYP enzyme — or yeast cells expressing multiple CYP enzymes.

The yeast cells may be immobilised upon the surface of the working electrode in a polymer film. This film could be fabricated from polymers including but not limited to: chitosan, polyacrylamide, polypyrrole, Nafion or PEDOT.

Chitosan may be chosen as the means of immobilisation as it is abundant, biocompatible, and is highly porous.

The addition of gold nanoparticles may be used to further improve the stability of the enzyme.

To improve the sensing characteristics of the film, nanoparticles could be incorporated within it alongside the yeast cells. Examples of such nanoparticles include, but are not limited to: gold nanoparticles, silver nanoparticles, platinum nanoparticles, copper nanoparticles, zinc oxide nanoparticles, nickel oxide nanoparticles, copper oxide nanoparticles, carbon nanoparticles, carbon nanotubes, graphene nanosheets, or any combination thereof.

The film could also be coated with a single layer or multiple layers of polymeric membrane material. Examples of such materials include, but are not limited to: polythene, Nafion, Teflon and cellulose.

Sensors formed in accordance with the present invention may demonstrate one or more of:
a limit of detection of 100 ng/ml down to 1 ng/ml, 0.1 ng/ml or 0.01 ng/ml (for example 49 ng/ml)
a linear range of 0 - 1.4 µg/ml
responds to changes of propofol concentration within one minute.

A further aspect provides an enzyme-based electrochemical propofol sensor that avoids issues of electrode fouling.

The present invention also provides a solution-phase propofol detection system for real-time monitoring of blood propofol concentration during general anaesthesia, comprising a real-time, point-of-care, blood propofol concentration measurement sensor and an analyte recovery system, allowing for continuous, real-time propofol monitoring without the need for drawing blood.

The analyte recovery system may comprise a molecular exchange means such as a microdialysis probe.

Some embodiments utilise microdialysis as a method of sampling because it can be automated, is continuous and, with the appropriate sensor, allows real-time online monitoring. Furthermore, this technique which involves the passage of analytes of interest (e.g. propofol) down a concentration gradient from blood to a perfusate separated by a thin semi-permeable membrane, means only the free fraction of the drug is sampled. Making it more applicable to monitor the pharmacologically relevant drug concentration. This therefore imposes more demand on the analytical method as generally only 2% (the free fraction) of the total drug concentration is available to be measured. Equivalent analytical ranges therefore become 0.04 - 0.1 mg/L for anaesthesia, and 0.01 - 0.3 mg/L for sedation, i.e. a range of 0.01 -0.1 mg/L, or 10 - 100 µg/L, (ng/mL) assuming 100% efficiency for drug recovery using microdialysis. However, as the target for monitoring is real-time, high microdialysis perfusion flow rates are needed which typically means lower recoveries and not uncommonly 1-10% meaning the target range could be as low as 0.1 - 1 µg/L (ng/mL).

Some embodiments provide or relate to a selective propofol biosensor that can be used in a microdialysis based sampling system.

A further aspect provides a propofol biosensor. A point-of-care, real-time blood propofol concentration measurement sensor comprising such a biosensor is also provided.

Sensors may be integrated into technologies to enable automated and continuous measurement.

Propofol sensors may, for example, be used in two ways: as an online sensor connected to a microdialysis device (as already discussed); or as a stand-alone sensor for inclusion in a biochemical analyser such as a blood gas analyser.

In the first approach, online sensor, it may be advantageous to align the electrodes within cells in such a way as to enhance performance when used in a continuous flow manner. Reference and/or counter electrodes may be arranged aligned to the direction of flow.

The sensor may detect the analyte by employing any appropriate electrochemical measurement technique, including but not limited to: amperometry, cyclic voltammetry, differential pulse voltammetry, square-wave voltammetry and coulometry.

In some embodiments an enzyme-based propofol sensor is provided that avoids issues of electrode fouling by converting the propofol into a quinone/quinol redox pair which can be detected via simple electrochemistry.

The sensor may, for example, respond to changes of propofol concentration within approximately 60 seconds, have a limit of detection of approximately 49 ng/ml and a linear response between approximately 0 and 1.4 µg/ml.

Sensors, methods and systems formed in accordance with the principles of the present invention can represent an important step in providing an effective means of point-of-care, real-time monitoring of the blood concentration of propofol for general anaesthesia patients.

A further aspect provides an electrochemical propofol sensor based upon the enzyme cytochrome P450 2B6.

The enzyme may, for example, be expressed within deactivated yeast cells (or other cells as described herein).

In some embodiments the yeast cells are in turn immobilised within, for example, a chitosan film containing, for example, gold nanoparticles upon the surface of a screen-printed electrode, for example.

The sequence of the electrochemical reaction in the sensor may be: reduction occurs before the oxidation.

The sensor may comprise a single sensor measuring the reduction current of 2,6-diisopropylquinone.

Some embodiments use two sensors in sequence with respect to a flow of the perfusate, the first measuring the reduction current of 2,6-diisopropylquinone and the second measuring the oxidation current of 2,6-diisopropylquinol.

Sequential sensors may be co-located on a single device or consist of two separate devices in sequence.

One embodiments of the present invention provides a propofol sensor based on deactivated yeast cells expressing cytochrome P450 2B6 immobilised within a chitosan membrane upon a graphite screen-printed electrode functionalised with a carbon nanotube/graphene oxide/iron oxide nanoparticle nanocomposite. In the presence of the relevant cofactors, the enzyme catalyses the conversion of propofol into a quinone/quinol redox pair, allowing for the simple electrochemical detection of propofol without any resultant electrode fouling. This sensor has a limit of detection of 7 ng/ml, is capable of detecting propofol in serum-like solutions and demonstrates a linear response over the therapeutic range of propofol. The sensor has been shown to demonstrate good selectivity toward a number of common perioperative drugs and has been shown to be stable after at least one week of storage.

Further aspects and embodiments are listed below, by way of example, in the following numbered paragraphs.

1. An enzyme-based electrochemical propofol sensor, the sensor being based on the enzyme cytochrome P450 2B6.

2. A sensor as claimed in paragraph 1, in which the sensor is based on an electrode.

3. A sensor as claimed in paragraph 2, in which the sensor is based on a screen-printed electrode.

4. A sensor as claimed in any preceding paragraph, comprising deactivated, permeabilised yeast, expressing cytochrome P450 2B6.

5. A sensor as claimed in any preceding paragraph, comprising deactivated yeast cells expressing the enzyme cytochrome P450 2B6 immobilised, alongside gold nanoparticles, within a chitosan film upon the surface of a screen-printed electrode

6. A sensor as claimed in any preceding paragraph integrated into technology to enable automated and continuous measurement.

7. A point-of-care, real-time blood propofol concentration measurement sensor comprising a sensor as claimed in any of paragraphs 1 to 5.

8. A solution-phase propofol detection system for real-time monitoring of blood propofol concentration during general anaesthesia, comprising a real-time, point-of-care, blood propofol concentration measurement sensor and an analyte recovery system, allowing for continuous, real-time propofol monitoring without the need for drawing blood.

9. A system as claimed in paragraph 8, in which the analyte recovery system comprises a molecular exchange means.

10. A system as claimed in paragraph 8 or paragraph 9, in which the analyte recovery system comprises a microdialysis probe.

11. A blood propofol concentration measurement sensor.

12. A sensor as claimed in paragraph 1, providing discrete measurement of propofol

13. A sensor as claimed in paragraph 1 or paragraph 2, forming part of a blood-gas analyser.

14. A sensor as claimed in paragraph 1, providing direct electrochemical measurement of propofol.

15. A sensor as claimed in any preceding claim, in which the sensor is enzymatic.

16. An enzyme-based electrochemical sensor for the detection of propofol.

17. A sensor as claimed in any preceding paragraph, in which the sensor is based on a one or more members of the cytochrome P450 group of enzymes.

18. A sensor as claimed in any preceding paragraph, in which the sensor is based on the enzyme cytochrome P450 2B6.

19. A sensor as claimed in any of paragraphs 15 to 18, in which enzymatic action converts propofol into a quinone/quinol redox pair.

20. A propofol detection system for point-of-care measurement of blood propofol concentration during general anaesthesia, comprising a blood-gas analyser having a solid-phase, enzymatic propofol concentration measurement sensor.

21. A solid-phase propofol detection system for real-time monitoring of blood propofol concentration during general anaesthesia, comprising a real-time, point-of-care, blood propofol concentration measurement sensor and an analyte recovery system, allowing for continuous, real-time propofol monitoring without the need for drawing blood.

22. A system as claimed in paragraph 11, in which the analyte recovery system comprises a molecular exchange means.

23. A system as claimed in paragraph 21 or paragraph 22, in which the analyte recovery system comprises a microdialysis probe

24. A propofol biosensor.

25. A sensor or system as claimed in any preceding paragraph, comprising a working electrode, a counter electrode and a reference electrode.

26. A sensor or system as claimed in any preceding paragraph, involving an electrochemical reaction with a redox step, in which oxidation occurs before the reduction.

27. An electrochemical propofol sensor based upon the enzyme cytochrome P450 2B6.

28. A sensor as claimed in paragraph 27, in which the enzyme is expressed within deactivated yeast cells.

29. A sensor as claimed in paragraph 28, in which the yeast cells are in turn immobilised within a chitosan film containing gold nanoparticles upon the surface of a screen-printed electrode.

30. A sensor as claimed in any of paragraphs 27 to 29, in which in the sequence of the electrochemical reaction reduction occurs before the oxidation.

31. A sensor as claimed in any of paragraphs 27 to 30, comprising a single sensor measuring the reduction current of 2,6-diisopropylquinone.

32. A sensor as claimed in any of paragraphs 27 to 30, comprising two sensors in sequence with respect to a flow of the perfusate, the first measuring the reduction current of 2,6-diisopropylquinone and the second measuring the oxidation current of 2,6-diisopropylquinol.

33. A sensor as claimed in paragraph 32, in which the sequential sensors are co-located on a single device or two separate devices in sequence.

34. A blood propofol enzymatic biosensor comprising a CYP enzyme, in which sensing occurs in the absence of direct electron transfer between an electrode and the CYP enzyme, using an indirect method via NADPH and electrochemically detecting the reaction products.

35. A real-time blood propofol monitoring system for TIVA-based anaesthesia.

36. A blood propofol monitoring system for TIVA-based anaesthesia.

37. A real-time, continuous propofol detector for a clinical setting.

Different aspects and embodiments of the invention may be used separately or together.

Further particular and preferred aspects of the present invention are set out in the accompanying independent and dependent claims. Features of the dependent claims may be combined with the features of the independent claims as appropriate, and in combination other than those explicitly set out in the claims.

Examples of aspects and embodiments of the present invention are shown in the accompanying drawings.

In the following description, all orientational terms, such as upper, lower, front, rear, radially and axially, are used in relation to the drawings and should not be interpreted as limiting on the invention or its connection to a closure.

Example embodiments are described below in sufficient detail to enable those of ordinary skill in the art to embody and implement the systems and processes herein described. It is important to understand that embodiments can be provided in many alternate forms and should not be construed as limited to the examples set forth herein.

Accordingly, while embodiments can be modified in various ways and take on various alternative forms, specific embodiments thereof are shown in the drawings and described in detail below as examples. There is no intent to limit to the particular forms disclosed. On the contrary, all modifications, equivalents, and alternatives falling within the scope of the appended claims should be included. Elements of the example embodiments are consistently denoted by the same reference numerals throughout the drawings and detailed description where appropriate.

The terminology used herein to describe embodiments is not intended to limit the scope. The articles “a,” “an,” and “the” are singular in that they have a single referent, however the use of the singular form in the present document should not preclude the presence of more than one referent. In other words, elements referred to in the singular can number one or more, unless the context clearly indicates otherwise. It will be further understood that the terms “comprises,” “comprising,” “includes,” and/or “including,” when used herein, specify the presence of stated features, items, steps, operations, elements, and/or components, but do not preclude the presence or addition of one or more other features, items, steps, operations, elements, components, and/or groups thereof.

Unless otherwise defined, all terms (including technical and scientific terms) used herein are to be interpreted as is customary in the art. It will be further understood that terms in common usage should also be interpreted as is customary in the relevant art and not in an idealized or overly formal sense unless expressly so defined herein.

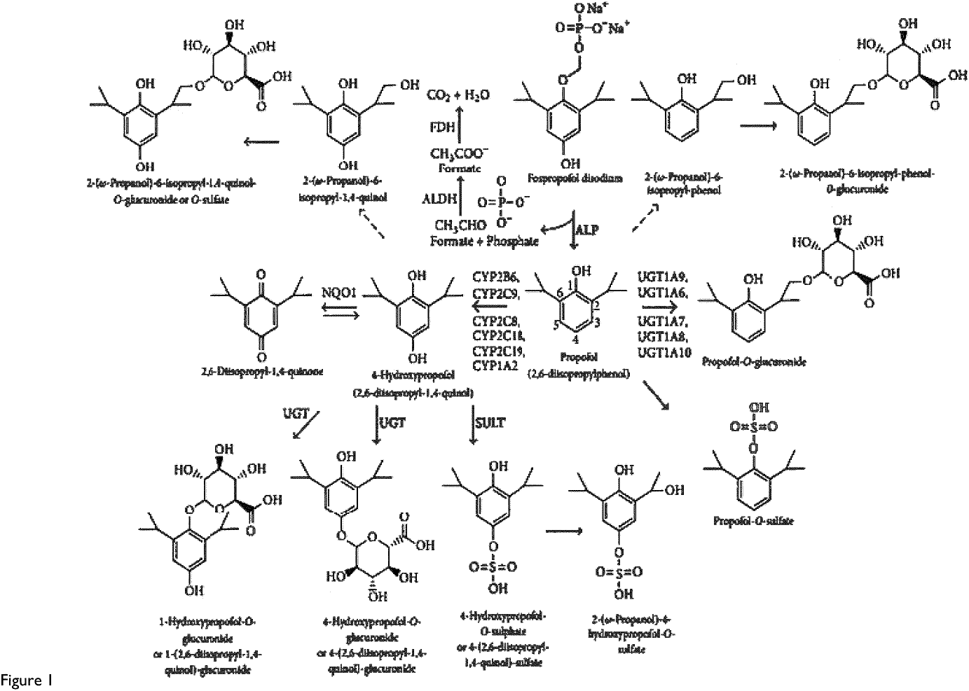

FIG. 1 shows metabolic pathways of propofol and fospropofol. Dashed arrows represent minor routes and both metabolites can undergo glucuronide and sulfate conjugation. SULT: sulfotransferase; UGT: UDP-glucuronosyltransferase; ALDH: aldehyde dehydrogenase; ALP: alkaline phosphatase; NQOI: diaphorase; CYP: cytochrome P450.

An electrochemical propofol sensor based upon the enzyme cytochrome P450 2B6 is described.

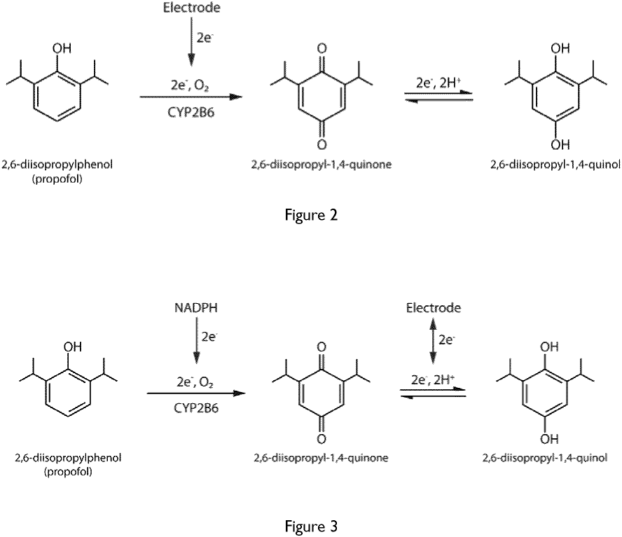

Some embodiments immobilise CYP2B6 enzyme at an electrode surface to provide a sensitive, real-time measurement of propofol. In the presence of oxygen, propofol undergoes a hydroxylation reaction to form a redox pair of 2,6-diisopropyl-1,4-quinone and 2,6-diisopropyl-1,4-quinol (FIG. 2 - adapted from Shioya et al., 2011) catalysed in the active site of the enzyme through two single electron reduction steps. Enzyme activity will draw electrons from the electrode resulting in a measured current proportional to the propofol concentration. As a result, cofactors would not be required.

Attempts to immobilise cytochrome P450 2B6 in this manner resulted in electrodes for which any electrochemical signal attributable to the enzyme rapidly diminished to the point that they were no longer discernible from the noise floor. It was eventually concluded that this was the result of enzyme denaturation due to the inherent instability of cytochrome P450 2B6. Many different immobilisation techniques including NHS/EDC linkages, diazonium linkages, multi-layer films, conductive polymer films and chitosan films were attempted in order to improve the enzyme stability, but were somewhat unsuccessful. Consequently, indirect detection may be favoured over direct methods. One such indirect method utilises CypExpress, deactivated yeast cells containing recombinant human CYP2B6, which are in turn immobilised within a chitosan film containing gold nanoparticles upon the surface of a screen-printed electrode. In the presence of the cofactor NADP⁺ the enzyme will convert propofol into a quinone/quinol redox pair. This redox pair can be detected electrochemically without resulting in electrode fouling, thus enabling the simple, direct and rapid measurement of propofol concentration. Chitosan was chosen as the means of immobilisation as it is abundant, biocompatible, and is highly porous. The addition of gold nanoparticles may be used to further improve the stability of the enzyme (Zhao, 2011; Gherardi, 2019).

In some embodiments the enzyme is expressed within deactivated yeast cells, which are in turn immobilised within a chitosan film containing gold nanoparticles upon the surface of a screen-printed electrode. Cytochrome P450 2B6 is one of the principle enzymes responsible for metabolising propofol within the human body. In the presence of the cofactor NADP⁺ the enzyme will convert propofol into a quinone/quinol redox pair (FIG. 3 - reaction mechanism for conversion of propofol into quinone/quinol redox pair by cytochrome P450 2B6. NADP+ acts as the electron source for the enzyme reaction and the electrode is used to detect the reaction products. Adapted from Shioya et al., 2011). The NADP⁺ acts as an electron source, allowing the enzyme to catalyse the conversion of propofol. Unlike direct electrochemical oxidation, the conversion of propofol in this manner will not lead to polymerisation and therefore will not cause electrode fouling. The resultant redox pair can be detected electrochemically, thus enabling the simple and rapid measurement of the propofol concentration. Chitosan was chosen as the means of immobilisation as it is abundant, biocompatible, and highly porous. The addition of gold nanoparticles is to aid electron transfer between the electrode and the deactivated yeast cells. In this embodiment the sensor has a non-limiting linear response over the range of approximately 0 - 1.4 µg/ml and a detection limit of approximately 49 ng/ml.

In one embodiment a CypExpress/gold nanoparticle/chitosan film was prepared by mixing CypExpress suspension (25 mg/ml in phosphate buffer, pH 7), gold nanoparticle suspension and a 1% chitosan solution (1% acetic acid) in a ratio of 1:1:2 by volume, drop-casting 1 µl onto the electrode surface and allowing to dry. The electrodes used are screen-printed three-electrode cells consisting of a 1 mm diameter graphite working electrode, a graphite counter electrode and a silver/silver chloride pseudo-reference electrode.

In some embodiments the sequence of the electrochemical reaction (i.e. positive peak before negative) is of importance for the sensor i.e. it has been found that reduction must occur before the oxidation. A device may comprise either a single sensor measuring the reduction current of 2,6-diisopropylquinone or two sensors in sequence with respect to the flow of the perfusate, the first measuring the reduction current of 2,6-diisopropylquinone and the second measuring the oxidation current of 2,6-diisopropylquinol. These sequential sensors may either be co-located on a single device or consist of two separate devices in sequence. The sequential measurement may offer superior discrimination from interfering compounds as while other compounds may be electroactive at one of the two voltages (positive or negative), it is less likely that they will be electroactive at both. Additionally, there are generally fewer interfering compounds in the vicinity of the negative voltages of the oxidation peak.

### MATERIALS AND METHODS

### Materials

All materials were supplied by Sigma-Aldrich and used as supplied. β-Nicotinamide adenine dinucleotide phosphate sodium salt hydrate (NADP⁺) and D-glucose 6-phosphate dipotassium salt hydrate (G6P) were dissolved in 10 mM phosphate buffered saline (PBS), pH 7.4, to produce solutions in which the concentration of each was 50 µg/ml. Unless stated otherwise, this is the testing solution for the electrochemical measurements described in section 2.4. 2,6-diisopropylphenol (97%) was diluted in dimethyl sulfoxide (99%) to create a 10 mM stock solution. This stock solution was further diluted with the NADP⁺/G6P solution to produce propofol solutions of varying concentrations as required for the tests described herein.

### Apparatus

The screen-printed electrodes used in these experiments were purchased from BVT Technologies. They constitute a three-electrode cell consisting of a 1 mm diameter graphite working electrode, a graphite counter electrode and a silver/silver chloride (Ag/AgCl) pseudo-reference electrode. All measurements were performed using a PalmSens EmStat³ potentiostat.

### Electrode Preparation

The electrodes were pre-treated by immersing them in a 10 mM K₃[Fe(CN)₆], 1 M KNO₃ solution and performing cyclic voltammetry between -0.6 and +0.8 V at a scan rate of 100 mV/s for a total of 10 cycles.

Gold nanoparticles were produced using a standard sodium citrate reduction technique. Briefly, 10 mg of gold chloride hydrate (HAuCl₄) (99.995%) were dissolved in 20 ml of deionised water and brought to boiling point under magnetic stirring. Trisodium citrate dihydrate (99%) was dissolved in deionised water to produce a 2.5% (w/v) solution and 1 ml of this solution was added to the HAuCl₄ solution and the mixture kept at boiling point for 5-10 minutes until it had undergone a colour change to deep red, before being allowed to cool to room temperature.

CypExpress 2B6 (a deactivated, permeabilised yeast, expressing cytochrome P450 2B6) was suspended in phosphate buffer (pH 7) at a concentration of 25 mg/ml. This suspension was mixed with the gold nanoparticle solution and a 1 % (w/v) chitosan solution (1% acetic acid) in a ratio of 1:1:2 by volume. 1 µl of this mixture was drop-cast onto the working electrode of the screen-printed electrodes and left to dry at 4° C. Once dry, the electrodes were immersed in 10 mM PBS for 30 minutes at room temperature, before being re-dried and stored at 4° C. until use.

### Electrochemical Measurement

Cyclic voltammetry measurements were performed by depositing 50 µl of propofol solutions of varying concentration upon the functionalised electrodes and cycling the potential between -0.8 and + 1.0 V at a rate of 100 mV/s.

Chronoamperometry measurements were performed by immersing the functionalised electrodes in 20 ml of 50 µg/ml NADP⁺ and G6P solution (10 mM PBS) and measuring the current at +0.5 V. The solution was stirred with a magnetic stirrer and 60 µl of 1 mM propofol solution were injected into the solution at regular intervals.

Control measurements were carried out by performing chronoamperometry as above in PBS solutions without NADP⁺ or G6P, and injecting 20 µl of 1 mM propofol solution at regular intervals. These experiments were carried out three times using the same electrode. Between each run the electrode was rinsed with 10 mM PBS, dried and stored at 4° C. overnight.

### RESULTS AND DISCUSSION

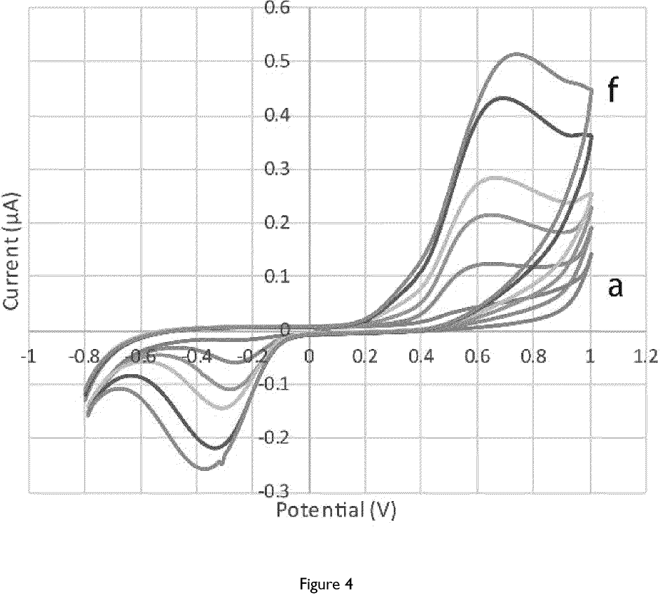

Cyclic voltammetry with varying propofol concentration (FIG. 4 - cyclic voltammograms of solutions of propofol concentrations: a) 0.18, b) 0.89, c) 1.78, d) 2.67, e) 3.57 and f) 4.47 µg/ml, all solutions contain 50 µg/ml NADP⁺ and G6P, 10 mM PBS. Scan rate is 100 mV/s. Potentials are vs. Ag/AgCl.) shows concentration dependant peaks at approximately + 0.6 V and -0.25 V. This behaviour is as would be expected for a quinone/quinol redox pair (Quan et al., 2007), with the peaks corresponding to the reduction of 2,6-diisopropylquinone and the oxidation of 2,6-diisopropylquinol respectively. This tells us that the enzyme within the yeast cells is converting propofol to its products as expected.

It has been found that oxidation of the quinol only occurs after reduction of the quinone. Monitoring at -0.2 V alone would provide no signal.

The performance of the sensor was assessed by immersing it in a 10 mM PBS solution containing 50 µg/ml of NADP⁺ and glucose-6-phosphate and performing amperometry at +0.6 V. The solution was spiked with a 1 mM propofol solution (10 mM PBS, 10% dimethyl sulfoxide) at regular intervals.

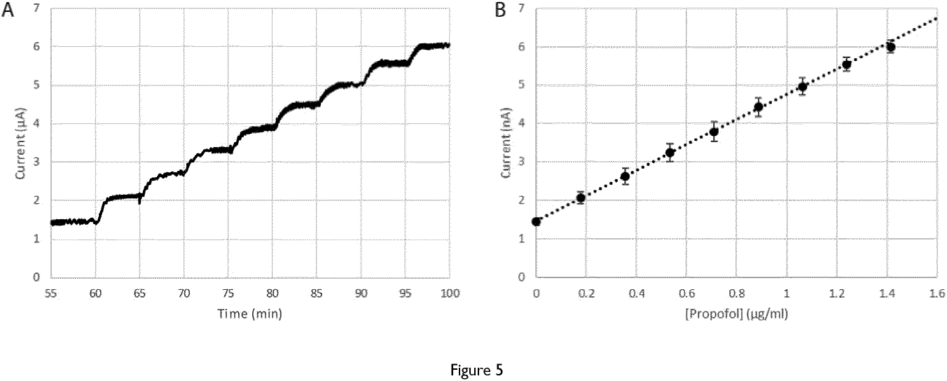

FIG. 5 - A: Chronoamperometric response of functionalised electrode with successive injections of propofol solution. Potential is +0.5 V vs. Ag/AgCl. Solution is 10 mM PBS containing 50 µg/ml NADP⁺ and 50 µg/ml G6P. B: Mean of the plateau current against propofol concentration. The error bars represent three standard deviations. Baseline correction has been applied.

Chronoamperometry measurements show a clear increase in current with each addition of propofol solution (FIG. 5A). The responses are fast, reaching a plateau in approximately 60 seconds, and stable throughout the experiment. Plotting the average plateau current against the propofol concentration (FIG. 5B) shows that the sensor produces a clear response to varying propofol concentration across the therapeutic range of 0.25 - 4 µg/ml (Langmaier et al., 2011). The response is linear -between 0 and 1.4 µg/ml, the sensitivity is 4.2 nA/µg/ml/mm² and the limit of detection is 49 ng/ml, well below the lower end of the therapeutic range.

The limit of detection (LoD) was calculated using the equation: LoD = 3.3 × σ_{low}/gradient, where σ_{low} is the standard deviation at a low propofol concentration.

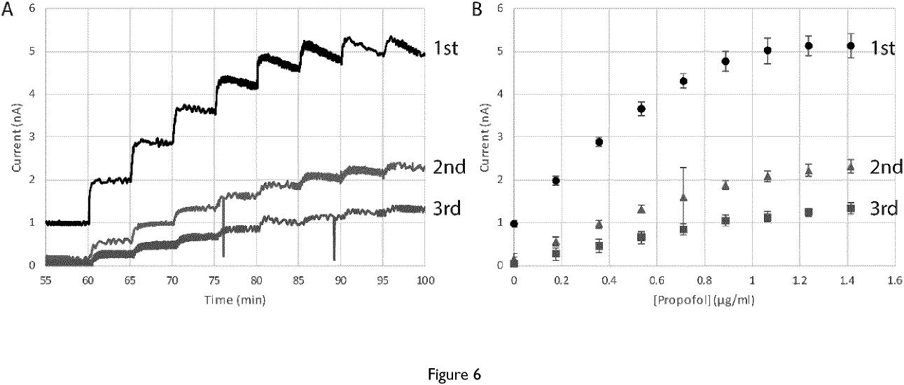

FIG. 6 - A: Chronoamperometric response of electrode with successive injections of propofol solution in the absence of NADP⁺ and G6P for the first, second and third repetitions of the same electrode on successive days. Potential is +0.5 V vs. Ag/AgCl, solution is 10 mM PBS. B: Mean of the plateau current against the propofol concentration. The error bars represent three standard deviations.

FIG. 6A shows the result of the chronoamperometry measurements in the absence of NADP⁺ and G6P. The increases in current after the addition of propofol are the result of the direct electrochemical oxidation of the propofol, which in the absence of NADP⁺ and G6P will not have been converted by the enzyme. In the first run it can be seen that after the fourth injection of propofol (corresponding to a concentration of approximately 0.7 µg/ml) there is a clear trend of decreasing current with time, suggestive of electrode fouling.

This is even more apparent in FIG. 6B in which it can be seen that the current response plateaus at approximately 1 µg/ml of propofol, a value that is comfortably within the linear range of the sensor as discussed above. Repeated measurements, performed using the same electrode on successive days, show a similar response but at significantly reduced sensitivities. The third run with the electrode has a sensitivity that is 22% that of the first run. These results are clear evidence of fouling of the electrode by propofol oxidation, fouling that does not occur in the presence of NADP⁺ and G6P, where the propofol is converted by the enzyme. Similar experiments performed with the addition of NADP⁺ and G6P result in the sensor showing a sensitivity on the third run that is approximately 95% of that on the first run (not shown).

To improve the performance of the sensor, the electrode surface may be functionalised with nanocomposites. Various materials have been investigated, but the most promising are nanocomposites combining carbon nanotubes (CNT) and graphene oxide (GO) decorated with metal oxide nanoparticles, specifically copper oxide nanoparticles (CuONP) and iron oxide nanoparticles (FeONP).

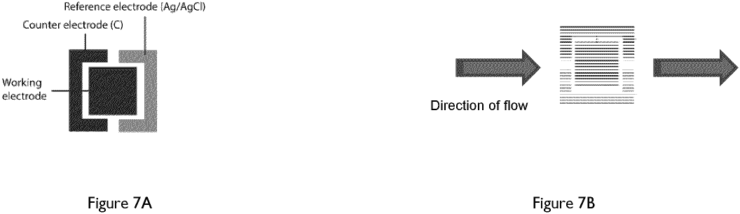

FIG. 7A shows a sensor formed in accordance with the present invention and including a working electrode, a counter electrode and a reference electrode.

Reference and/or counter electrodes may be arranged aligned to the direction of flow, as illustrated in FIG. 7B.

FIGS. 8 to 17 show further embodiments of the present invention.

FIG. 8 is an illustration of functionalisation of an electrode surface with nanocomposite.

Planar carbon electrode with a film containing gold nanoparticles and a single type of yeast cells expressing a single CYP enzyme. Three electrode cell with a macroscale working electrode.

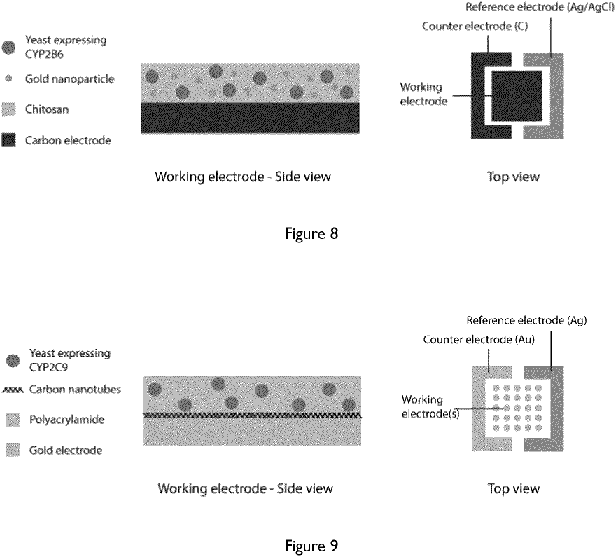

FIG. 9 - Planar gold electrode, functionalised with a single layer of carbon nanotubes, with a film containing a single type of yeast cells expressing a single CYP enzyme. Three electrode cell with a working electrode consisting of a microarray.

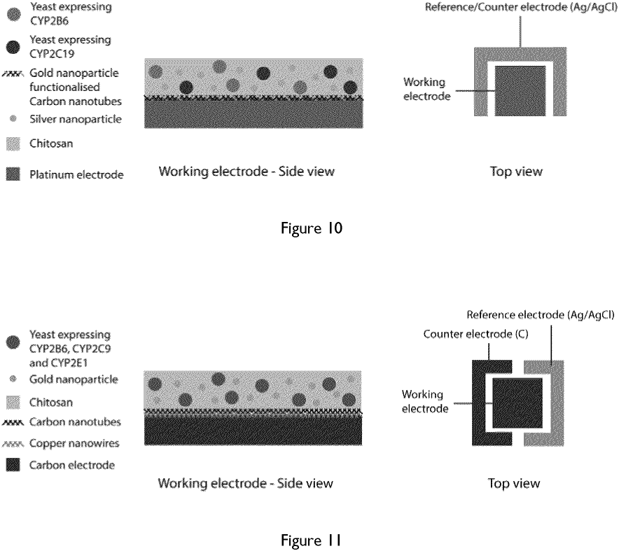

FIG. 10 - Planar platinum electrode, functionalised with a composite layer of gold nanoparticle functionalised carbon nanotubes, with a film containing silver nanoparticles and two types of yeast cells each expressing a single CYP enzyme. Two electrode cell with a macroscale working electrode and a combined counter/reference electrode.

FIG. 11 - Planar carbon electrode, functionalised with two layers of different nanomaterials, with a film containing gold nanoparticles and a single type of yeast cells expressing multiple CYP enzymes. Three electrode cell with a macroscale working electrode.

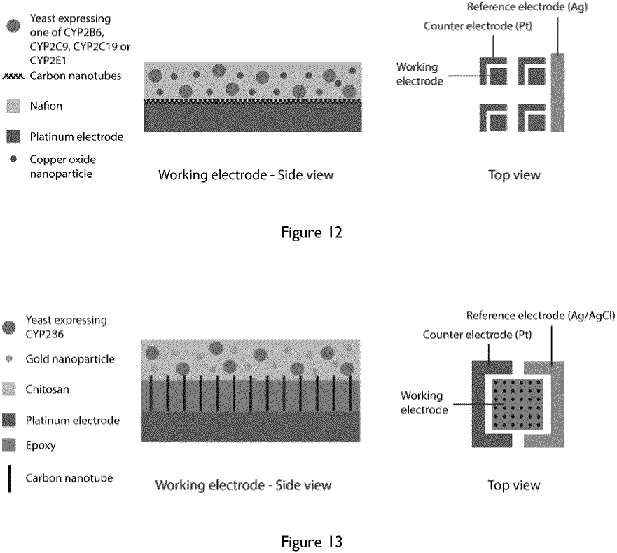

FIG. 12 - Planar platinum electrode, functionalised with a single layer of carbon nanotubes, with a film containing copper oxide nanoparticles and a single type of yeast cells expressing a single CYP enzyme. Multi-electrode device with multiple microscale working electrodes, each with a yeast expressing a different CYP enzyme, each with an associated counter electrode and sharing a common reference electrode (three electrode cells).

FIG. 13 - Planar platinum electrode, functionalised with vertically aligned carbon nanotubes partially encapsulated within and epoxy so as to form a nanoelectrode array. Film containing gold nanoparticles and a single type of yeast cells expressing a single CYP enzyme. Three electrode cell with a working electrode consisting of a nanoelectrode array.

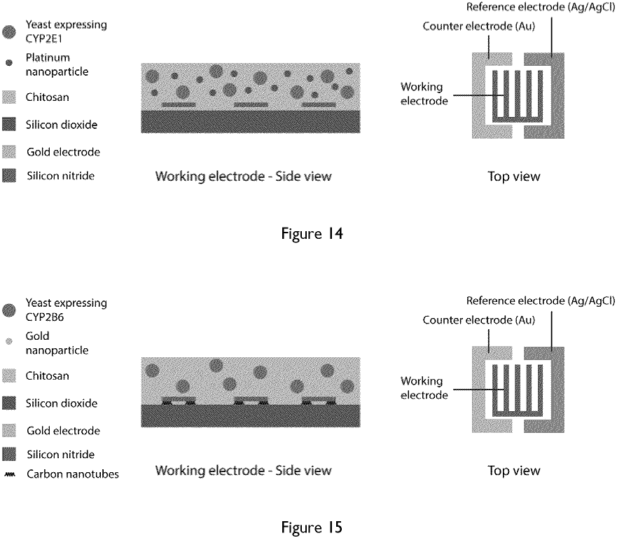

FIG. 14 - Nanostrip electrode, with a silicon nitride passivation layer, with a film containing platinum nanoparticles and a single type of yeast cells expressing a single CYP enzyme. Three electrode cell with a nanostrip working electrode.

FIG. 15 - Gold nanostrip electrode coated with a conductive network of carbon nanotubes and a silicon nitride passivation layer. Film containing gold nanoparticles and a single type of yeast cells expressing a single CYP enzyme. Three electrode cell with a nanostrip working electrode.

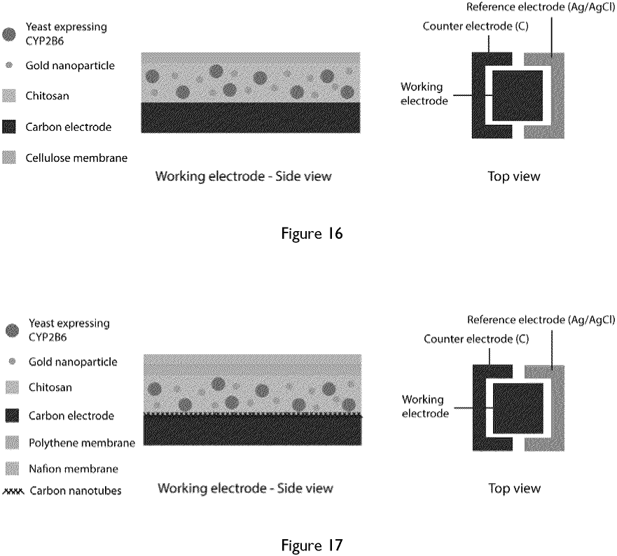

FIG. 16 - Planar carbon electrode with a film containing gold nanoparticles and a single type of yeast cells expressing a single CYP enzyme. The film is coated with a cellulose membrane. Three electrode cell with a macroscale working electrode.

FIG. 17 - Planar carbon electrode, functionalised with a single layer of carbon nanotubes, with a film containing gold nanoparticles and a single type of yeast cells expressing a single CYP enzyme. The film is coated with a bilayer membrane consisting of a layer of polythene and a layer of Nafion. Three electrode cell with a macroscale working electrode.

The performance of nanocomposite functionalised sensors was assessed using the same amperometric measurements described previously.

The sensitivities and limits of detection of each variant of nanocomposite are shown in Table 1.

Table 1 shows the average sensitivities and limits of detection (LoD) for multiple replicates of CNT/GO, CNT/GO/CuONP and CNT/GO/FeONP electrodes (one example of each of which are shown in Error! Reference source not found.). As already discussed, electrodes prepared using metal-oxide nanoparticle decorated graphene oxide show significant increased sensitivity compared to electrodes prepared using non-decorated graphene oxide, with FeO nanoparticles resulting in the greatest improvement. However, the LoD of CNT/GO/CuONP is higher than that of CNT/GO, a fact that can be attributed to increased noise. However, the LoD for CNT/GO/FeONP electrodes is 7.0 ± 1.2, which is approximately half that of the CNT/GO electrodes, a significant improvement. In a previous publication [REF] we showed that the LoD for a sensor consisting of the type of enzyme film described here on a bare carbon screen printed electrode was 49 ng/ml. Therefore, it can be seen that these carbon nanocomposite functionalised electrodes offer significant improvements in sensitivity for propofol detection, with composites of carbon nanotubes and iron-oxide nanoparticle decorated graphene oxide offering the greatest improvement.

TABLE 1

Sensitivities and limits of detection for electrodes functionalised with various different nanomaterials

| Electrode functionalisation | Sensitivity (nA/µg/ml/mm²) | LoD, pre- processing (ng/ml) | LoD, post processing (ng/ml) |
|---|---|---|---|
| CNT/GO | 12.2 ± 0.5 | 18.9 ± 0.6 | 14.2 ± 0.5 |
| CNT/GO/CuO NP | 18.6 ± 3.8 | 48.6 ± 15.3 | 19.9 ± 8.1 |
| CNT/GO/FeO NP | 29.9 ± 6.4 | 17.7 ± 6.6 | 7.0 ± 1.2 |

TABLE 2

Sensitivity and limit of detection for sensors functionalised with nanocomposites

| Electrode functionalisation | Sensitivity (nA/µg/ml/mm²) | Limit of detection (ng/ml) |
|---|---|---|
| No nanocomposite | 4.1 ± 0.2 | 67 ± 7 |
| CNT/GO | 12.2 ± 0.5 | 14.2 ± 0.5 |
| CNT/GO/CuONP | 18.6 ± 3.8 | 19.9 ± 8.1 |
| CNT/GO/FeONP | 29.9 ± 6.4 | 7.0 ± 1.2 |

The nanocomposite functionalised electrodes present a significant improvement compared to the non-functionalised electrode, with the CNT/GO/FeONP composites offering the greatest improvement. This improvement in performance is a result of a combination of increased surface area, improved electron transfer and catalytic effects of the nanocomposite. A moving average filter (with a ten second bin) is applied to the current response as a means of noise reduction, providing additional improvement to the limit of detection.

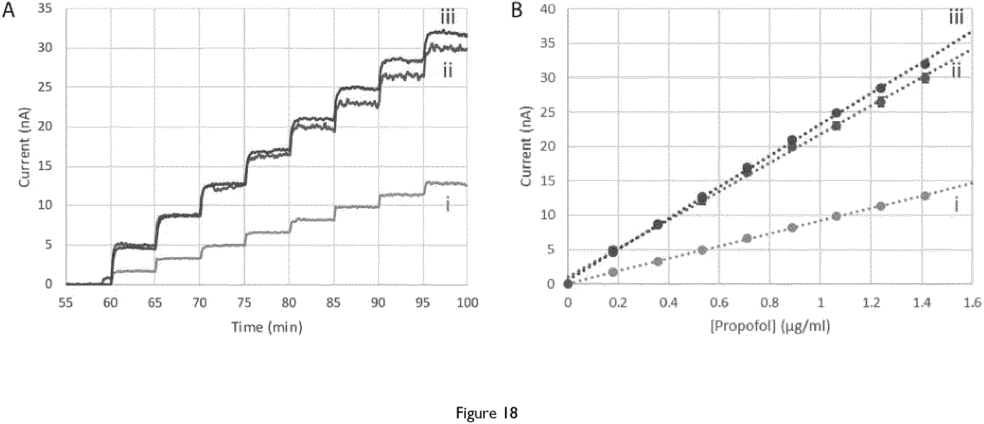

FIG. 18: A) Amperometric response of sensor to successive injections of propofol solution for i) CNT/GO, ii) CNT/GO/CuONP, and iii) CNT/GO/FeONP functionalised sensors B) Mean of plateau current against resultant propofol concentration. Potential is +0.5 V vs. screen printed Ag/AgCl. Solution is 10 mM PBS containing 50 µg/ml NADP⁺ and 50 µg/ml G6P. Error bars represent three standard deviations. Baseline correction and moving average filter have been applied.

18A shows the results of amperometry measurements for electrodes functionalised with CNT/GO (i), CNT/GO/CuONP (ii), and CNT/GO/FeONP (iii) for an increasing propofol concentration (propofol solution is injected every five minutes). The average plateau currents versus the resultant propofol concentration is shown in 18B. It can be seen that all three sensors produce clear increases in current with increasing propofol concentration. These responses are fast, occurring within one minute, and stable throughout the experiment. In all cases the current response is linear with respect to propofol concertation over the range investigated.
CNT - Carbon nanotube
CuONP - Copper oxide nanoparticle
FeONP - Iron oxide nanoparticle
GO - Graphene oxide

The sensitivity of the electrodes prepared using metal-oxide decorated graphene oxide appears much greater than that of the sensor prepared using non-decorated graphene oxide. As discussed previously, the cyclic voltammetry results do not suggest improvements in terms of surface area or electron transfer are achieved through the inclusion of the metal-oxide nanoparticles, suggesting that the improvements insensitivity are the result of catalytic properties of the metal-oxide nanoparticles.

It is clear that the nanocomposite functionalised electrodes present a significant improvement compared to the non-functionalised electrode, with the CNT/GO/FeONP composites offering the greatest improvement. This improvement in performance is a result of a combination of increased surface area, improved electron transfer and catalytic effects of the nanocomposite. A moving average filter (with a ten second bin) is applied to the current response as a means of noise reduction, providing additional improvement to the limit of detection.

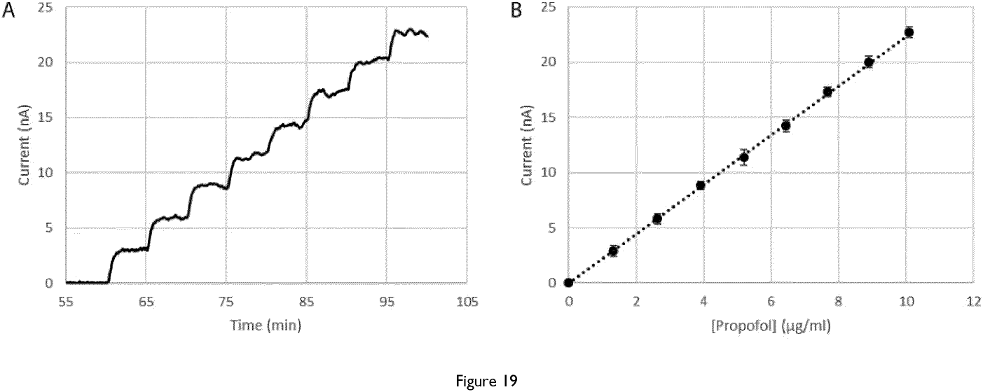

FIG. 19: A) Amperometric response of CNT/GO/FeONP functionalised sensor to successive injections of propofol solution in serum-like solution (5 wt% BSA, 10 mM PBS), B) Mean of plateau current against resultant propofol concentration. Potential is +0.5 V vs. screen printed Ag/AgCl. Solution is 10 mM PBS containing 50 µg/ml NADP⁺ and 50 µg/ml G6P. Error bars represent three standard deviations. Baseline correction has been applied.

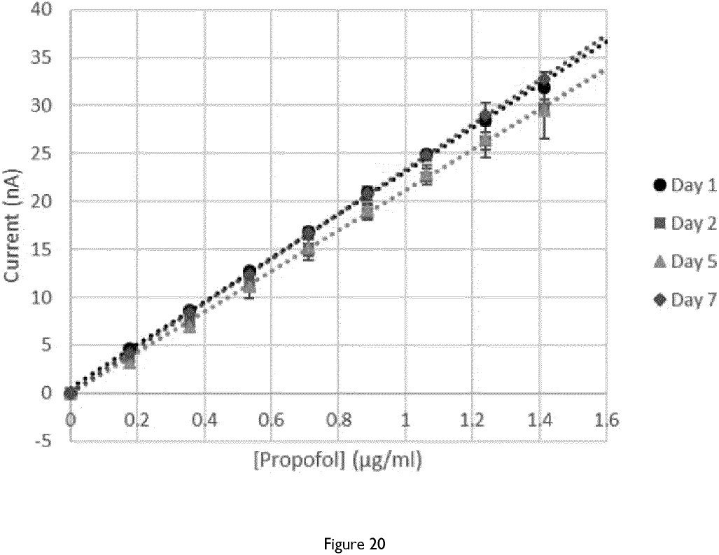

In order to assess the performance of the sensor in conditions closer to physiological conditions, solutions were prepared containing 5 wt% of bovine serum albumin (BSA), 137 mM NaCl, 2.7 mM KCl and 10 mM phosphate buffer (pH 7.4), in addition to 50 µg/ml NADP⁺ and glucose-6-phosphate. These solutions are considered “serum-like” as they have physiological salinity (Opoku-Okrah, 2015), pH (Brørs, 1985) and albumin concentration (Kim, 2020). In these solutions the sensor produces a linear current response across the therapeutic range of propofol (1-10 µg/ml) (Rengenthal, 1999). The sensitivity is 3.1 ± 0.2 nA/µg/ml/mm² and the limit of detection is 143 ± 27 ng/ml (FIG. 20). The sensitivity is lower and the limit of detection is higher than that obtained in PBS, which is to be expected as the majority of the propofol will be bound to the albumin, as discussed previously, and therefore the sensor is only detecting the free-fraction. However, this limit of detection is still comfortably below the lower end of the therapeutic range.

FIG. 20: Current response against propofol concentration for CNT/GO/FeONP sensors tested 1, 2, 5 and seven days after fabrication.

The nanocomposite sensors have been shown to produce consistent results up to seven days of storage after fabrication (FIG. 20), with what little variation in sensitivity and limit of detection observed accountable for by inter-device variation. The sensors were stored at 4° C. between fabrication and testing. Longer term shelf-life tests (over periods of months) are currently being carried out.

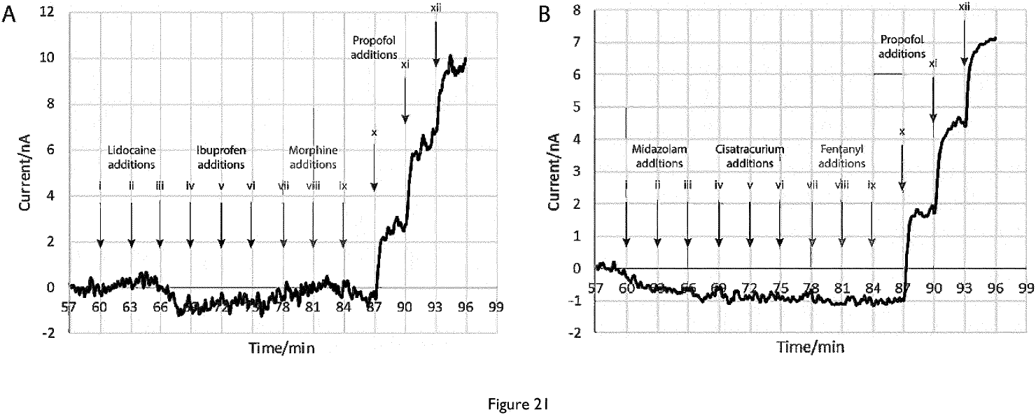

In order to assess the specificity of the sensor towards potential interfering perioperative drugs, amperometry measurements were performed as previously, injecting various different drugs at regular intervals (FIG. 21). The sensor produces no discernible response to the common perioperative drugs: lidocaine (Altermatt, 2012), ibuprofen (Rainsford, 2009), morphine (Pinky, 2020), midazolam (Wong, 1991), cistricurium besilate (Guo, 2017) and fentanyl (Peng, 1999). Each drug is injected at the appropriate concentration to produce a final concentration that shares the same approximate proportionality to the final propofol concentration as their expected therapeutic ranges (Regenthal, 1999).

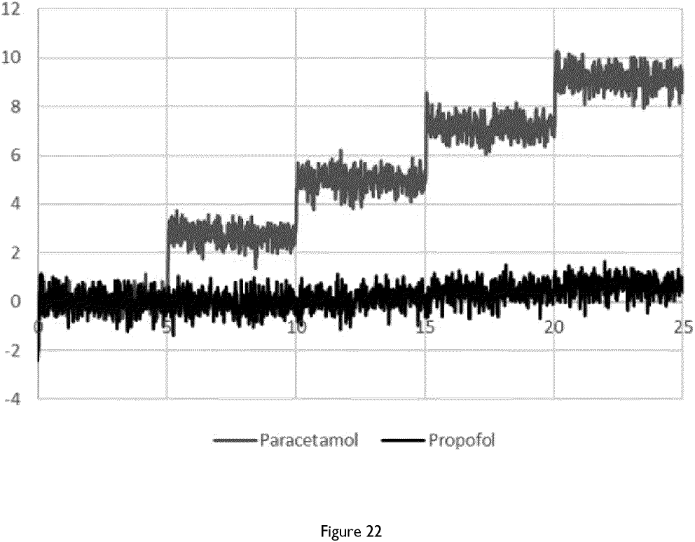

FIG. 22: Amperometric response of CNT/GO/FeO NP/enzyme functionalised electrode with successive injections of A: lidocaine solution (i-iii; 0.23, 0.47 and 0.70 µg/ml respectively), ibuprofen solution (iv-vi; 0.21, 0.41 and 0.62 µg/ml respectively), morphine solution (vii-ix; 0.0028, 0.0057 and 0.0085 µg/ml respectively) and propofol solution (x-xii; 0.18, 0.35 and 0.53 µg/ml respectively) B: Midazolam solution (i-iii; 0.0033, 0.0065 and 0.0097 µg/ml respectively), cisatracurium besilate solution (iv-vi; 0.12, 0.25 and 0.37 µg/ml respectively), fentanyl solution (vii-ix; 0.0018, 0.0035 and 0.0053 µg/ml respectively) and propofol solution (x-xii; 0.18, 0.35 and 0.53 µg/ml respectively). Potential is +0.5 V vs. screen printed Ag/AgCl. Solution is 10 mM PBS containing 50 µg/ml NADP⁺ and 50 µg/ml G6P.

One potential perioperative drug that does result in interference is the commonly used anti-inflammatory drug paracetamol (Colsoul, 2019), as a result of the similarities in the chemical structure between it and propofol. Various attempts to mitigate this potential source of interference were investigated including Nafion membranes and molecularly imprinted polymers. One relates to the fact that paracetamol can be oxidised at a lower potential than propofol or the products of the enzymatic reaction. This can be seen in FIG. 22 which shows amperometric measurements at +0.25 V for electrodes functionalised with CNT/GO/FeONP nanocomposite (but without enzyme film) with successive injections of both propofol and paracetamol solutions. It is clear that at this potential, the electrode produces a response for paracetamol but not for propofol, allowing the possibility of a second electrode at a lower potential selectively measuring paracetamol concentration, which can then be subtracted from the final signal. Dual-potential methodology may be used to account for potential paracetamol interference.

FIG. 22: Current response for CNT/GO/FeONP functionalised electrodes (no enzyme film) for amperometric measurement at + 0.25 V with successive injections of propofol (black) and paracetamol (red) solutions.

### Nanoparticle Synthesis

Metal oxide nanoparticles were synthesised using methods adapted from Jamzad et al.

Bay leaf extract was prepared by grinding 20 g of dried bay leaves to a powder using a mortar and pestle. The powdered bay leaves were then added to 200 ml of deionised water and stirred at 90° C. for 10 minutes. The resultant solution was filtered and then centrifuged to remove any remaining plant material. This bay leaf extract solution was stored at 4° C. until required, and used within four weeks.

For copper oxide and iron oxide decorated graphene oxide (GO), GO was added to 0.1 M metal salt solution (either FeCl₃ or CuCl₂) at a concentration of 1 mg/ml and sonicated for 30 minutes to disperse the GO. This dispersion was added to bay leaf extract solution at a ratio of 1:1 and left at room temperature overnight to allow metal oxide nanoparticles to form.

The metal oxide nanoparticle-decorated graphene oxide was then extracted from solution by centrifuging at 5000 rpm for 15 minutes and washed by re-suspending in de-ionised water and re-centrifuging three times. The metal oxide nanoparticle-decorated graphene oxide was then suspended in de-ionised water once again and added to MWCNTs and the mixture sonicated for 1 hour to produce a dispersion with 2 mg/ml MWCNT and 1 mg/ml GO. The dispersion is then diluted with de-ionised water to a concentration of 0.1 mg/ml MWCNT and 0.05 mg/ml GO.

### Electrode Functionalisation

The MWCNT/GO/MONP dispersions described are sonicated for 1 hour to ensure maximal dispersion. 1 µl of the dispersion is then drop-cast onto the working electrode of the SPEs and allowed to evaporate before a second 1 µl is drop-cast and dried in the same manner. The electrodes are then rinsed with deionised water to remove any unbound nanomaterials.

Preparation of the enzyme film has been described previously. Briefly, CypExpress 2B6 is suspended in a phosphate buffer (pH7) at a concentration of 25 mg/ml and this suspension is mixed with a gold nanoparticle solution and a 1% chitsosan solution (1% acetic acid) in a ratio of 1:1:2 by volume. 1 µl of this mixture is deposited on the WE of the SPEs and left to dry at 4° C. Once dry, the electrodes are immersed in 10 mM PBS for 30 minutes and then dried in air at room temperature. The functionalised electrodes are stored at 4° C. until use.

### Results

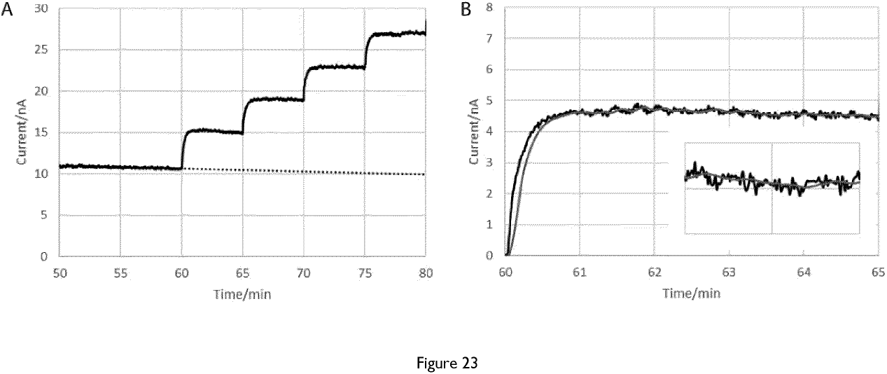

FIG. 23 - Examples of A: Baseline correction, and B: smoothing by moving average filter. Inset - zoomed in section of between 63 and 64 minutes.

In order to counteract the effects of drift and noise that are common for sensors of this type, some simple signal processing was applied. Firstly, baseline correction was applied by performing a liner fit to the current response from the five minutes prior to the first injection of propofol solution and correcting all of the data so that it is measured relative to this baseline. An example of this is shown in 23A in which the raw data is depicted by the solid line and the calculated baseline is depicted by the dotted line. The sensor drift is evidenced by the slight downward trend of the baseline.

Secondly, smoothing was performed by applying a moving average filter to the current data with a bin size of ten seconds. The bin size was decided upon as being a reasonable compromise between degree of smoothing and induced time-lag. An example of this is shown in 23B in which the raw data (depicted by the black line) is plotted alongside smoothed data (depicted by the blue line). The reduction in the noise by the filter is clearly evident.

Although illustrative embodiments of the invention have been disclosed in detail herein, with reference to the accompanying drawings, it is understood that the invention is not limited to the precise embodiments shown and that various changes and modifications can be effected therein by one skilled in the art without departing from the scope of the invention.

### REFERENCES

Cussonneau, X., De Smet, E., Lantsoght, K., Salvi, J.-P., 2007, J. Pharma. Biomed. Anal., 44, 680-682. Vishwananthan, K., Stewart, J., T., 1999, J. Chromatogr. Relat. Technol., 22, 923-931.

Hikiji, W., Kudo, K., Usumoto, Y., Tsuji, A., Ikeda, N., 2010, J. Anal. Toxicol., 34, 389-393.

Lee, S. Y., Parl, N.-H., Jeong, E.-K., Wi, J.-W., Kim, C.-J., Kim, J. Y., In, M. K., Hong, J., 2012, J. Chromatogr. B, 900, 1-10.

Liu, B., Pettigrew, D. M., Bates, S., Laitenberger, P. G., Troughton, G., 2012, J. Clin. Monit. Comput., 26, 29-36.

Diao,J., Wang, T., Li, L., 2019, Royal Soc. Open Sci., 6, 181753.

Anon. (2016) Insights into CYP2B6-mediated drug-drug interactions. Acta Pharmaceutica Sinica. B [online]. 6 (5), pp.413-425.

Ouchi, K. and Sugiyama, K. (2015) Required propofol dose for anesthesia and time to emerge are affected by the use of antiepileptics: prospective cohort study. BMC Anesthesiology [online]. 15 (1), pp.34. Smits, A., Annaert, P. and Allegaert, K. (2017) Biomarkers of propofol metabolism in neonates: the quest beyond ontogeny. [online].

Dinis-Oliveira, R.J. (2018) Metabolic Profiles of Propofol and Fospropofol: Clinical and Forensic Interpretative Aspects. BioMed Research International [online]. 2018 pp.6852857-16.

Altermatt, F R et al., Evaluation of the effect of intravenous lidocaine on propofol requirements during total intravenous anaesthesia as measured by bispectral index. Br. J. Anaesth., 2012 Jun; 108(6): 979-983. Brørs, O and Jacobsen, S, pH lablility in serum during equilibriam dialysis. Br. J. Clin. Pharmac., 1985 Jul; 20: 85-88.

Colsoul, M-L et al., Long-term stability of an infusion containing paracetamol, alizapride, ketorolac and tramadol in glass bottles at 5±3° C. Eur. J. Hosp. Pharm., 2019 Jun; 27: e74-e78.

Cussonneau, X et al., A rapid and simple HPLC method for the analysis of propofol in biological fluids, J. of Pharm. Biomed. Anal., 2007 Nov; 44: 680-682.

Eisenried, A et al., Determination of total and unbound propofol in patients during intensive care sedation by ultrafiltration and LC-MS/MS. J Pharm Biomed Anal., 2016 Jul 15; 126:148-55.

Fan, S-Z et al., Propofol concentration monitoring in plasma or whole blood by gas chromatography and high-performance liquid chromatography. Anesth. Analg., 1995 Jul; 81(1):175-8.

Gherardi, F et al., Immobilized enzymes on gold nanoparticles: From enhanced stability to cleaning of heritage textiles. ACS App. Bio Mater., 2019 Oct; 2: 5136-5143.

Guo, J et al., Pharmacokinetics and pharamcodynamics of cisatracurium in patients undergoing surgery with two henodilution methods. J. Clin. Anesth., 2017 May; 38: 75-80.

Kim, J W et al., Serum albumin and beta-amyloid deposition in the human brain. Neurology, 2020 Jul; 95: e815-e826.

Kwak, J-H, et al., Determination of propofol glucuronide from hair sample by using mixed mode anion exchange cartridge and liquid chromatography tandem mass spectrometry. J Chromatogr., 2016 Mar;1015-1016:209-21 3.

Liu, B et al., Performance evaluation of a whole blood propofol analyser. J Clin. Monit. Comput., 2012 Feb;26(1):29-36.

Mazoit, J X and Samii K, Binding of propofol to blood components: implications for pharmacokinetics and for pharmacodynamics. Br. J. Clin. Pharmacol., 1999 Jan; 47(1): 35-42.

Miekisch, W. et al., Assessment of propofol concentrations in human breath and blood by means of HS-SPME-GC-MS. Clin Chim Acta. 2008 Sep;395(1-2):32-7.

Opoku-Okrah, C et al., Changes in potassium and sodium concentrations in stored blood., Pan African Medical J., 2015 Mar; 20: 236.

Peng, P W H et al., A review of the use of fentanyl analgesia in the management of acute pain in adults. Anesthesiol., 1999 Feb; 90: 576-599.

Peng, L., et al., Electrochemistry of cytochrome P450 2B6 on electrodes modified with zirconium dioxide nanoparticles and platin components. Electroanal., 2008 Mar; 20(7): 803-807.

Pinky, A et al., Review on the progress in electrochemical detection of morphine based on different modified electrodes. J. Electrochem. Soc., 2020 Feb; 167: 037559.

Preethichandra, D et al. Performance enhancement of polypyrrole based nano-biosensors by different enzyme deposition techniques, Smart Sensors, Measurement and Instrumentation, 2019; 29: 213-229. Quan, M et al., Voltammetry of quinones in unbuffered aqueous solution: reassessing the roles of proton transfer and hydrogen bonding in the aqueous electrochemistry of quinones. J. Am. Chem. Soc., 2007 Oct; 129(42): 12847-12856.

Rainey, F. et al., Toward feedback controlled anesthesia: automated flow analytical system for electrochemical monitoring of propofol, Electroanalysis, 2014 Apr; 26(6):1295-1303.

Rainsford K D, lbuprofen:pharmacology, efficacy and safety. Inflammopharmacol., 2009 Dec; 17: 275-342. Regenthal, R et al., Drug levels: therapeutic and toxic serum/plasma concentrations of common drugs. J. Clin. Monit. Comput. 1999 Dec; 15: 529-544.

Schneider, E and Clark, D, Cytochrome P450 (CYP) enzymes and the development of CYP biosensors. Biosens. Bioelectron., 2013 Jan 15; 39(1):1-13.

Shioya, N., et al., Green urine discoloration due to propofol infusion: A case report. Case Rep. Emerg. Med., 2011:242514.

Vaiano, F et al., LC-MS/MS and GC-MS methods in propofol detection: Evaluation of the two analytical procedures., Forensic Sci. Int., 2015 Nov; 256:1-6.

Wong, H Y et al., Dose-finding study of intramuscular Midazolam preanesthetic medication in the elderly. Anesthesiol., 1991 Apr; 74: 675-679.

Yang, X et al., Modelling analysis of electrode fouling during electrolysis of phenolic compounds. Electrochimica Acta, 2013 Jan; 94:259-268.

Zhao, S et al., A gold nanoparticle-mediated enzyme bioreactor for inhibitor screening by capillary electrophoresis. Anal. Biochem., 2011 Apr; 411(1): 88-93.

CN102262082B, Optical fiber sensor-molecular imprinting online detection method for detecting propofol in biological samples and optical fiber sensor-molecular imprinting online detection device. Filed 2011-07-12.

M. Jamzad and M. K. Bidkorpeh, “Green synthesis of iron oxide nanoparticles by the aqueous extract of Laurus nobilis L. leaves and evaluation of the antimicrobial activity,” Journal of Nanostructures in Chemistry, vol. 10, pp. 193-201, 2020.

S. Fakhari, M. Jamzad and H. K. Fard, “Green synthesis of zinc oxide nanoparticles: a comparison,” Green Chemistry Letters and Reviews, vol. 12, no. 1, pp. 19-24, 2019.

1. A blood propofol concentration measurement sensor, wherein the sensor is enzymatic, and wherein the sensor is based on one or more members of the cytochrome P450 group of enzymes.

2. The blood propofol concentration measurement sensor of claim 1, providing discrete measurement of propofol.

3. The blood propofol concentration measurement sensor of claim 1, forming part of a blood-gas analyser.

4. The blood propofol concentration measurement sensor of claim 1, providing direct electrochemical measurement of propofol.

5. (canceled)

6. An enzyme-based electrochemical sensor for the detection of propofol, wherein the sensor is based on one or more members of the cytochrome P450 group of enzymes.

7. (canceled)

8. The enzyme-based electrochemical sensor of claim 6 in which the sensor is based on the enzyme cytochrome P450 2B6.

9. The enzyme-based electrochemical sensor of claim 6, in which enzymatic action converts propofol into a quinone/quinol redox pair.

10. A propofol detection system for point-of-care measurement of blood propofol concentration during general anaesthesia, comprising the enzyme-based electrochemical sensor of claim 6.

11. The propofol detection system of claim 10 comprising an analyte recovery system, the analyte recovery system allowing for continuous, real-time propofol monitoring without the need for drawing blood.

12. The propofol detection system of claim 11, in which the analyte recovery system comprises a molecular exchange means.

13. The propofol detection system of claim 11, in which the analyte recovery system comprises a microdialysis probe.

14. (canceled)

15. The enzyme-based electrochemical sensor of claim 6, comprising a working electrode, a counter electrode and a reference electrode.

16. The enzyme-based electrochemical sensor of claim 6, comprising an electrochemical reaction with a redox step, in which in a sequence of the electrochemical reaction, an oxidation occurs before the a reduction.

17. (canceled)

18. The enzyme-based electrochemical sensor of claim 6, in which the enzyme is expressed within deactivated yeast cells.

19. The enzyme-based electrochemical sensor of claim 18, in which the yeast cells are in turn immobilized within a chitosan film containing gold nanoparticles upon the surface of a screen-printed electrode.

20. The enzyme-based electrochemical sensor of claim 6, comprising an electrochemical reaction with a redox step in which in a sequence of the electrochemical reaction, a reduction occurs before an oxidation.

21. The enzyme-based electrochemical sensor of claim 6, comprising a single sensor measuring a reduction current of 2,6-diisopropylquinone.

22. The enzyme-based electrochemical sensor of claim 6, comprising two sensors in sequence with respect to a flow of a perfusate associated with the detection of propofol, the first measuring a reduction current of 2,6-diisopropylquinone and the second measuring an oxidation current of 2,6-diisopropylquinol.

23. The enzyme-based electrochemical sensor of claim 22, in which the two sensors in sequence are co-located on a single device or two separate devices in sequence.

24. The enzyme-based electrochemical sensor of claim 6 comprising a CYP enzyme, in which sensing occurs in the absence of direct electron transfer between an electrode and the CYP enzyme, using an indirect method via NADPH and electrochemically detecting the reaction products.

25. (canceled)
